(19) United States
(12) Patent Application Publication
(10) Pub. No.: US 2015/0051213 A1
(43) Pub. Date: Feb. 19, 2015

(54) NOVEL SALTS OF SITAGLIPTIN

(71) Applicants: Suresh Babu Jayachandra, Gurgaon (IN); Jigar Bhaskarbhai Shah, Bharuch (IN); Sailu Chiguru, Karimnager (IN)

(72) Inventors: Suresh Babu Jayachandra, Gurgaon (IN); Jigar Bhaskarbhai Shah, Bharuch (IN); Sailu Chiguru, Karimnager (IN)

(21) Appl. No.: 14/129,893

(22) PCT Filed: Jun. 26, 2012
(86) PCT No.: PCT/IB2012/053234

§ 371 Date: Oct. 27, 2014

(30) Foreign Application Priority Data
Jun. 30, 2011 (IN) 1867/DEL/2011
Aug. 16, 2011 (IN) 2323/DEL/2011

(51) Int. Cl. C07D 487/04; C07C 55/14; C07C 63/24; C07D 213/79; C07C 65/10; C07C 53/126

(52) U.S. Cl. CPC C07D 487/04; C07C 65/10; C07C 53/126; C07C 63/24; C07D 213/79; C07C 55/14
USPC 514/249; 544/350

(57) ABSTRACT

The present invention provides sitagliptin 4-methylsalicylate, sitagliptin myristate, sitagliptin isophthalate, sitagliptin isonicotinide, sitagliptin adipate, their polymorphic form, processes for their preparation and pharmaceutical compositions thereof.

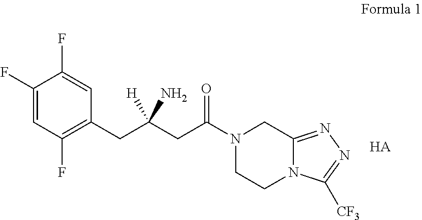

### FIELD OF THE INVENTION

The present invention provides novel salts of sitagliptin, its polymorphic form, processes for their preparation and pharmaceutical compositions thereof.

### BACKGROUND OF THE INVENTION

Sitagliptin dihydrogen phosphate monohydrate of Formula A, an orally-active inhibitor of the dipeptidyl peptidase-4 (DPP-4) enzyme, chemically designated as 7-[(3R)-3-amino-1-oxo-4-(2,4,5-trifluorophenyl)butyl]-5,6,7,8-tetrahydro-3-(trifluoromethyl)-1,2,4-triazolo[4,3-c]pyrazine phosphate (1:1) monohydrate is indicated as an adjunct to diet and exercise to improve glycemic control in adults with type 2 diabetes mellitus.

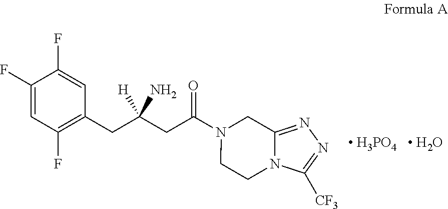

U.S. Pat. No. 6,699,871 (hereinafter “the '871 patent”), in particular Example 7, provides a process for the preparation of sitagliptin base and its hydrochloride salt. A list of pharmaceutically acceptable salts is generally included in the '871 patent.

U.S. Pat. No. 7,326,708 provides a process for the preparation of sitagliptin dihydrogen phosphate monohydrate.

PCT Publication WO 2005/072530 provides a process for the preparation of crystalline salts of sitagliptin with hydrochloric acid, benzene sulfonic acid, p-toluene sulfonic acid, D- and L-tartaric acid and (1S)-(+)- and (1R)-(−)-10-camphorsulfonic acid.

PCT Publication WO 2005/030127 (hereinafter “PCT '127”) provides a process for the preparation of sitagliptin dihydrogen phosphate anhydrate Form IV which involves heating sitagliptin dihydrogen phosphate monohydrate at 120° C. for about 2 hours or by heating the sitagliptin dihydrogen phosphate monohydrate above 58° C. for about 8 hours.

PCT '127 also provides a process for the preparation of sitagliptin dihydrogen phosphate anhydrate Form I by heating sitagliptin dihydrogen phosphate anhydrate Form IV at a temperature above 140° C. for about 1 hour. According to this publication, Form IV is metastable and converts to the crystalline monohydrate slowly under ambient conditions and rapidly under high relative humidity (98%) at room temperature.

PCT Publication WO 2005/020920 provides a process for the preparation of crystalline anhydrate Form I, crystalline desolvated anhydrate Form II, crystalline anhydrate Form III, crystalline ethanol solvate of sitagliptin dihydrogen phosphate. It also provides a process for the preparation of mixture of sitagliptin dihydrogen phosphate anhydrate Form I and anhydrate Form III.

PCT Publication WO 2006/033848 provides a process for the preparation of crystalline sitagliptin dihydrogen phosphate monohydrate and amorphous sitagliptin dihydrogen phosphate.

PCT Publication WO 2007/035198 provides a process for the preparation of dodecylsulfate salt of sitagliptin.

PCT Publication WO 2008/000418 provides a process for the preparation of anhydrous sitagliptin hydrochloride in amorphous form.

PCT Publication WO 2009/120746 provides processes for the preparation of crystalline form of sitagliptin phosphate, characterized by a powder XRD pattern with peaks at about 4.7, 13.5, 17.7, 18.3, and 23.7±0.2° 2θ and sitagliptin phosphate Form II.

U.S. Publication 2009/247532 provides processes for the preparation of polymorph Form V of crystalline sitagliptin phosphate and polymorph Form I of sitagliptin phosphate.

PCT Publication WO 2009/084024 provides a process for the preparation of R-sitagliptin dibenzyl-L-tartrate.

PCT Publication WO 2009/085990 provides a process for the preparation of crystalline anhydrate Form A of the dihydrogen phosphate salt of sitagliptin, crystalline sitagliptin sulfate, crystalline sitagliptin hydrobromide, crystalline sitagliptin methane sulfonate, crystalline sitagliptin acetate, crystalline sitagliptin benzoate, crystalline sitagliptin oxalate, crystalline sitagliptin succinate, crystalline sitagliptin mandelate, crystalline sitagliptin fumarate and crystalline sitagliptin lactate.

PCT Publication WO 2010/032264 provides a process for the preparation of crystalline Form 3 of sitagliptin, crystalline form of dibenzoyl-L-tartaric acid salt of sitagliptin, amorphous form of sitagliptin and anhydrous and hydrated crystalline form of phosphate salt of sitagliptin.

PCT Publication 2010/000469 provides a process for the preparation of sitagliptin hydrochloride Form I, sitagliptin hydrochloride Form II, sitagliptin fumarate Form I, sitagliptin fumarate Form II, sitagliptin malate, sitagliptin sulfate Form I, sitagliptin sulfate Form II, sitagliptin phosphate, sitagliptin succinate Form I and Form II, sitagliptin succinate Form III, sitagliptin lactate, sitagliptin glycolate, sitagliptin maleate Form I, sitagliptin maleate Form II, sitagliptin citrate, amorphous sitagliptin citrate, sitagliptin mesylate Form I and sitagliptin mesylate Form II.

PCT Publication WO 2010/012781 provides a process for the preparation of sitagliptin galactarate, sitagliptin hemi-L-malate, sitagliptin D-gluconate, sitagliptin succinate, sitagliptin hydrobromide, sitagliptin thiocyanate, sitagliptin oxalate, sitagliptin aspartate, sitagliptin ethanedisulfonate, sitagliptin pyroglutamate, sitagliptin glutarate, sitagliptin acetate, sitagliptin hydrochloride amorphous form, sitagliptin citrate amorphous form, sitagliptin hemicitrate amorphous form, sitagliptin glycolate amorphous form and sitagliptin malate amorphous form.

PCT Publication WO 2010/117738 provides a process for the preparation of crystalline Form S1 of sitagliptin sulfate, crystalline Form S2 of sitagliptin sulfate, crystalline Form S3 of sitagliptin sulfate, crystalline Form S4 of sitagliptin sulfate, crystalline Form S5 of sitagliptin sulfate, crystalline Form S6 of sitagliptin sulfate, crystalline Form S7 of sitagliptin sulfate, crystalline Form S8 of sitagliptin sulfate, crystalline Form D1 of sitagliptin (+)-dibenzoyl-tartrate, crystalline Form D2 of sitagliptin (+)-dibenzoyl-tartrate, crystalline Form F1 of sitagliptin fumarate, crystalline Form F2 of sitagliptin fumarate, crystalline Form M1 of sitagliptin (D)-(+)-malate, crystalline Form M2 of sitagliptin (D)-(+)-malate, crystalline Form I1 of sitagliptin L-malate, crystalline Form O1 of sitagliptin oxalate, crystalline Form O2 of sitagliptin oxalate, crystalline Form Q1 of sitagliptin quinate, crystalline Form U1 of sitagliptin succinate, crystalline Form E1 of sitagliptin acetate, crystalline Form A1 of sitagliptin maleate, crystalline Form N1 of sitagliptin (S)-mandelate, crystalline Form N2 of sitagliptin (S)-mandelate, crystalline Form N3 of sitagliptin (S)-mandelate, crystalline Form N4 of sitagliptin (S)-mandelate, amorphous sitagliptin mandelate, crystalline Form N5 of sitagliptin (R)-mandelate, crystalline Form N6 of sitagliptin (R)-mandelate, crystalline Form L1 of sitagliptin lactate, crystalline Form L2 of sitagliptin lactate, crystalline Form L3 of sitagliptin lactate, crystalline Form L4 of sitagliptin lactate and amorphous sitagliptin orotate.

PCT Publication WO 2010/092090 provides a process for the preparation of crystalline sitagliptin D-glucuronate, crystalline sitagliptin glutarate, crystalline sitagliptin hydrogen sulfate, crystalline sitagliptin L-lactate, crystalline sitagliptin oxalate, sitagliptin caprate, sitagliptin L-mandelate, crystals of sitagliptin ethanesulfonate.

PCT Publication WO 2010/122578 provides a process for the preparation of sitagliptin hydrogen phosphate monohydrate and sitagliptin mandalate.

PCT Publication WO 2011/025932 provides a process for the preparation of sitagliptin phosphate and sitagliptin hydrochloride.

PCT Publication WO 2011/060213 provides a process for the preparation of sitagliptin phosphate, sitagliptin formate and sitagliptin acetate.

PCT Publication WO 2011/018494 provides a process for the preparation of sitagliptin fumarate.

Journal of Medicinal Chemistry, 48(1), p. 141-151 (2005) provides a process for the preparation of sitagliptin hydrochloride and sitagliptin fumarate.

Several processes are known in the literature for making sitagliptin or a salt thereof, for example, PCT Publications WO 2011/049344, WO 2010/131025, WO 2010/078440, WO 2004/083212, WO 2006/065826, WO 2010/097420, WO 2004/080958, WO 2004/087650 and WO 2004/085661.

In the pharmaceutical industry, there is a constant need to identify the critical physicochemical parameters such as novel salts, novel polymorphic forms that affect the drug's performance, stability, etc., which may play a key role in determining a drug's market acceptance and success.

Since sitagliptin is an important therapeutic agent, developing other, hitherto unknown salts is of value to pharmaceutical science, especially in terms of having improved solubility, stability, excellent storage and handling stabilities, bioavailability, etc.

Polymorphism is commonly defined as the ability of any substance to have two or more different crystal structures. Drug substances may also encapsulate solvent molecules when crystallized. These solvates or hydrates are referred to as pseudo polymorphs.

Different polymorphs, pseudo polymorphs or the amorphous form differ in their physical properties such as melting point, solubility, etc. These can appreciably influence pharmaceutical properties such as dissolution rate and bioavailability. It is also economically desirable that the product is stable for extended periods of time without the need for specialized storage conditions.

It is therefore important to evaluate polymorphism of drug substances. Therefore, there is also strong need for developing various polymorphic forms of salts of sitagliptin.

### SUMMARY OF THE INVENTION

In one general aspect, the present invention provides sitagliptin 4-methyl salicylate.

In another general aspect, the present invention provides amorphous form of sitagliptin 4-methyl salicylate.

In another general aspect, the present invention provides sitagliptin myristate.

In yet another general aspect, the present invention provides crystalline form of sitagliptin myristate.

In another general aspect, the present invention provides sitagliptin isophthalate.

In yet another general aspect, the present invention provides crystalline form of sitagliptin isophthalate.

In another general aspect, the present invention provides sitagliptin isonicotinate.

In yet another general aspect, the present invention provides crystalline form of sitagliptin isonicotinate.

In another general aspect, the present invention provides sitagliptin adipate.

In yet another general aspect, the present invention provides crystalline form of sitagliptin adipate.

In another general aspect, the present invention provides a process for the preparation of a compound of Formula 1.

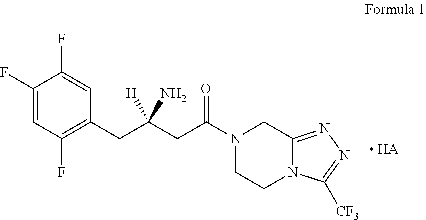

The process comprises: treating sitagliptin or its salt and HA, wherein HA is selected from the group consisting of 4-methylsalicylic acid, myristic acid, isophthalic acid, isonicotinic acid and adipic acid.

In another general aspect, the present invention provides the use of sitagliptin 4-methyl salicylate, sitagliptin myristate, sitagliptin isophthalate, sitagliptin isonicotinate or sitagliptin adipate for the preparation of sitagliptin, salts, solvates, or polymorphs thereof.

In yet another general aspect, the present invention provides a pharmaceutical composition comprising salt of sitagliptin 4-methyl salicylate, sitagliptin myristate, sitagliptin isophthalate, sitagliptin isonicotinate or sitagliptin adipate and a pharmaceutical acceptable carrier.

In another general aspect, the present invention provides a method of treating or preventing type 2 diabetes mellitus which comprises administering to a patient in need thereof a therapeutically effective amount of sitagliptin 4-methyl salicylate, sitagliptin myristate, sitagliptin isophthalate, sitagliptin isonicotinate or sitagliptin adipate and a pharmaceutical acceptable carrier.

### DETAILED DESCRIPTION OF THE INVENTION

The present invention provides various salts of sitagliptin. For example, the present invention provides sitagliptin 4-methyl salicylate. Sitagliptin 4-methyl salicylate of the present invention may be in amorphous form. Amorphous form of sitagliptin 4-methyl salicylate of the present invention may be characterized by an XRPD pattern substantially the same as depicted in FIG. 1. Amorphous form of sitagliptin 4-methyl salicylate of the present invention may be characterized by FTIR as depicted in FIG. 2.

The present invention also provides sitagliptin myristate. Sitagliptin myristate of the present invention may be in crystalline form. Crystalline form of sitagliptin myristate of the present invention may be characterized by an XRPD pattern substantially the same as depicted in FIG. 3. Crystalline form of sitagliptin myristate of the present invention may be characterized by FTIR as depicted in FIG. 4. The crystalline form of sitagliptin myristate has an XRPD pattern which may include interplanar spacing (d) values substantially at about 18.31, 9.21, 4.20, 4.19, and 3.71 Å. The crystalline form of sitagliptin myristate has an XRPD pattern which may further include interplanar spacing (d) values substantially at about 4.76, 4.68, 4.56, 4.43, 4.44, 4.30, 3.82, 3.49, 3.42, 3.36, and 3.18 Å. The crystalline form of sitagliptin myristate has an XRPD pattern with the following characteristic peak values (2θ) at about 4.82, 9.60, 21.11, 21.20, and 23.91±0.2°. The crystalline form of sitagliptin myristate has an XRPD pattern with the following additional characteristic peak values (2θ) at about: 18.63, 18.97, 19.46, 19.96, 20.06, 20.62, 23.26, 25.46, 25.99, 26.48, and 27.98±0.2°.

The present invention also provides sitagliptin isophthalate. Sitagliptin isophthalate of the present invention may be in crystalline form. Crystalline form of sitagliptin isophthalate of the present invention may be characterized by an XRPD pattern substantially the same as depicted in FIG. 5. Crystalline form of sitagliptin isophthalate of the present invention may be characterized by FTIR as depicted in FIG. 6. The crystalline form of sitagliptin isophthalate has an XRPD pattern which may include interplanar spacing (d) values substantially at about 3.49, 4.59, 3.91, 3.77, and 4.71 Å. The crystalline form of sitagliptin isophthalate has an XRPD pattern which may further include interplanar spacing (d) values substantially at about 23.73, 14.89, 7.03, 6.12, 5.80, 5.66, 5.51, 5.23, 5.16, 4.99, 4.78, 4.42, 4.32, 4.06, 3.99, 3.58, 3.34, 3.24, 3.13, 3.00, and 2.81 Å. The crystalline form of sitagliptin isophthalate has an XRPD pattern with the following characteristic peak values (2θ) at about 18.82, 19.35, 22.72, 23.59, and 25.54±0.2°. The crystalline form of sitagliptin isophthalate has an XRPD pattern with the following additional characteristic peak values (2θ) at about: 3.72, 5.94, 12.59, 14.48, 15.27, 15.67, 16.09, 16.95, 17.19, 17.76, 18.55, 20.10, 20.56, 21.90, 22.24, 24.86, 26.72, 27.56, 28.54, 29.73, and 31.84±0.2°.

The present invention also provides sitagliptin isonicotinate. Sitagliptin isonicotinate of the present invention may be in crystalline form. Crystalline form of sitagliptin isonicotinate of the present invention may be characterized by an XRPD pattern substantially the same as depicted in FIG. 7. Crystalline form of sitagliptin isonicotinate of the present invention may be characterized by FTIR as depicted in FIG. 8. The crystalline form of sitagliptin isonicotinate has an XRPD pattern which may include interplanar spacing (d) values substantially at about 7.95, 5.17, 3.99, 3.65, and 3.58 Å. The crystalline form of sitagliptin isonicotinate has an XRPD pattern which may further include interplanar spacing (d) values substantially at about: 15.84, 5.39, 5.32, 4.52, 4.16, 3.83, 3.81, 3.35, and 3.24 Å. The crystalline form of sitagliptin isonicotinate has an XRPD pattern with the following characteristic peak values (2θ) at about: 11.12, 17.14, 22.26, 24.41, and 24.85±0.2°. The crystalline form of sitagliptin isonicotinate has an XRPD pattern with the following additional characteristic peak values (2θ) at about: 5.58, 16.45, 16.66, 19.64, 21.34, 23.23, 23.36, 26.59 and 27.53±0.2°.

The present invention also provides sitagliptin adipate. Sitagliptin adipate of the present invention may be in crystalline form. Crystalline form of sitagliptin adipate of the present invention may be characterized by an XRPD pattern substantially the same as depicted in FIG. 9. Crystalline form of sitagliptin adipate of the present invention may be characterized by FTIR as depicted in FIG. 10. Crystalline form of sitagliptin adipate has an XRPD pattern which may include interplanar spacing (d) values substantially at about 4.70, 4.38, 4.00, 3.82, 3.77 and 3.71 Å. Crystalline form of sitagliptin adipate has an XRPD pattern which may further include interplanar spacing (d) values substantially at about 17.57, 10.99, 6.31, 5.50, 4.79, 4.74, 4.22, 3.95, 3.70, 3.62 and 3.33 Å. Crystalline form of sitagliptin adipate has an XRPD pattern with the following characteristic peak values (2θ) at about 18.90, 20.27, 22.20, 23.29, 23.63 and 23.97±0.2°. Crystalline form of sitagliptin adipate has an XRPD pattern with the following additional characteristic peak values (2θ) at about: 5.03, 8.05, 14.04, 16.13, 18.53, 18.72, 21.03, 22.50, 24.04, 24.60 and 26.74±0.2°.

The present invention provides a process for the preparation of a compound of Formula 1.

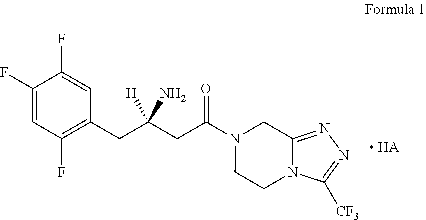

The process comprises: treating sitagliptin or its salt and HA, wherein HA is selected from the group consisting of 4-methylsalicylic acid, myristic acid, isophthalic acid, isonicotinic acid or adipic acid.

The sitagliptin or its salt prepared by any of the methods known in the art including those described in, for example, U.S. Pat. Nos. 6,699,871, 7,326,708, US Publication No. 2009/247532, PCT Publication Nos. WO 2010/131025, WO 2004/083212, WO 2006/065826, WO 2010/097420, WO 2004/080958, WO 2004/087650, WO 2004/085661, WO 2005/072530, WO 2005/030127, WO 2005/020920, WO 2007/035198, WO 2008/000418, WO 2009/120746, WO 2006/033848, WO 2009/085990, WO 2010/032264, WO 2010/000469, WO 2010/012781, WO 2010/117738, WO 2010/092090, WO 2010/122578 may be used as the starting material.

The sitagliptin or its salt prepared by any of the methods known in the art may be isolated or directly treated with HA.

The sitagliptin or its salt prepared by any of the methods known in the art before treatment with HA may be optionally clarified to remove foreign particulate matter or treated with activated charcoal to remove coloring and other related impurities in a suitable solvent. The solution of sitagliptin or its salt may be optionally concentrated to reduce the amount of solvent. The sitagliptin salt may optionally be converted to sitagliptin base before the treatment with HA.

Treating sitagliptin or its salt with HA may include adding, dissolving, slurrying, stirring or a combination thereof. Sitagliptin or its salt may be treated with HA directly or in the presence of a suitable solvent at a suitable temperature.

The term “solvent” includes any solvent or solvent mixture, including, for example, water, esters, alkanols, halogenated hydrocarbons, ketones, ethers, polar aprotic solvents, or mixtures thereof.

The esters may include one or more of ethyl acetate, n-propyl acetate, isopropyl acetate, and n-butyl acetate. Examples of alkanol include those primary, secondary and tertiary alcohols having from one to six carbon atoms. Suitable alkanol solvents include methanol, ethanol, n-propanol, isopropanol and butanol. Examples of halogenated hydrocarbons include dichloromethane, chloroform, and 1,2-dichloroethane. Examples of ketones include acetone, methyl ethyl ketone, and the like. Examples of ethers include diethyl ether, tetrahydrofuran, and the like. A suitable polar aprotic solvent includes one or more of N,N-dimethylformamide, N,N-dimethylacetamide, dimethylsulphoxide, acetonitrile and N-methylpyrrolidone.

Sitagliptin or its salt may be treated with HA at a temperature of about 30° C. to reflux temperature for a time period sufficient to complete the reaction, preferably for about 10 minutes to 6 hours.

After the completion of the reaction, the compound of Formula 1 can be isolated by the common isolation technique such as cooling, extraction, one or more of washing, crystallization, precipitation, filtration, filtration under vacuum, decantation and centrifugation, or a combination thereof.

The present invention also provides for the use of sitagliptin 4-methyl salicylate, sitagliptin myristate, sitagliptin isophthalate, sitagliptin isonicotinate or sitagliptin adipate for the preparation of sitagliptin, salts, solvates, or polymorphs thereof.

The compound of Formula 1 may be used for preparation of sitagliptin by contacting with a base. The base may be selected from group comprising of hydroxides, carbonates and bicarbonates of alkali and alkaline earth metals, ammonia, alkyl amines, hydrazine, and the like. Examples of hydroxides, carbonates and bicarbonates of alkali and alkaline earth metals may include lithium hydroxide, sodium hydroxide, potassium hydroxide, sodium carbonate, potassium carbonate, sodium bicarbonate or potassium bicarbonate. Examples of alkyl amines may include diethyl amine, triethyl amine or methyl diethyl amine. Sitagliptin thus obtained may be converted to salts, solvates, or polymorphs thereof.

The present invention also provides for a pharmaceutical composition comprising salt of sitagliptin 4-methyl salicylate, sitagliptin myristate, sitagliptin isophthalate, sitagliptin isonicotinate or sitagliptin adipate and a pharmaceutical acceptable carrier.

The present invention provides for a method of treating or preventing type 2 diabetes mellitus which comprises administering to a patient in need thereof a therapeutically effective amount of sitagliptin 4-methyl salicylate, sitagliptin myristate, sitagliptin isophthalate, sitagliptin isonicotinate or sitagliptin adipate and a pharmaceutical acceptable carrier.

### BRIEF DESCRIPTION OF THE FIGURES

FIG. 1 depicts the X-Ray Powder Diffractogram (XRPD) of sitagliptin 4-methyl salicylate prepared as per Example 1.

FIG. 2 depicts the Fourier-Transform Infra-red (FTIR) spectrum of sitagliptin 4-methyl salicylate prepared as per Example 1.

FIG. 3 and FIG. 3a depict the X-Ray Powder Diffractogram (XRPD) of sitagliptin myristate and the associated values, respectively, prepared as per Example 2.

FIG. 4 depicts the Fourier-Transform Infra-red (FTIR) spectrum of sitagliptin myristate prepared as per Example 2.

FIG. 5 and FIG. 5a depict the X-Ray Powder Diffractogram (XRPD) of sitagliptin isophthalate and the associated values, respectively, prepared as per Example 3.

FIG. 6 depicts the Fourier-Transform Infra-red (FTIR) spectrum of sitagliptin isophthalate prepared as per Example 3.

FIG. 7 and FIG. 7a depict the X-Ray Powder Diffractogram (XRPD) of sitagliptin isonicotinate and the associated values, respectively, prepared as per Example 4.

FIG. 8 depicts the Fourier-Transform Infra-red (FTIR) spectrum of sitagliptin isonicotinate prepared as per Example 4.

FIG. 9 and FIG. 9a depict the X-Ray Powder Diffractogram (XRPD) of sitagliptin adipate and the associated values, respectively, prepared as per Example 5.

FIG. 10 depicts the Fourier-Transform Infra-red (FTIR) spectrum of sitagliptin adipate prepared as per Example 5.

The X-ray powder diffractograms (XRPD) of the samples were determined by using Instrument: PANalytical, Mode: Expert PRO, Detector: Xcelerator, ScanRange: 3-40, Step size: 0.02, Range: 3-40 degree 2 theta, CuKα radiation at 45 kV.

FTIR of the samples was determined by using Instrument: Perkin Elmer, SCAN: 16 scans, Resolution: 4.0 cm⁻¹, potassium bromide pellet method.

While the present invention has been described in terms of its specific embodiments, certain modifications and equivalents will be apparent to those skilled in the art and are intended to be included within the scope of the present invention.

### EXAMPLES

### Example 1

### Preparation of Sitagliptin 4-Methyl Salicylate Salt

Sitagliptin base (1 g, 0.00245 mole) was charged in isopropyl alcohol (10 ml) at 25° C. to 32° C. 4-Methyl salicylic acid (0.37 g, 0.00245 mole) was charged at 25° C. to 32° C. The reaction mixture was heated to 50° C. and stirred for 2 hours at 50° C. The reaction mixture was then cooled to 25° C. to 32° C. and distilled under vacuum completely at 50° C. to obtain a solid. The solid was dried under vacuum at 40° C. for 16 hours to obtain the titled compound.

### Yield: 1 g

### Example 2

### Preparation of Sitagliptin Myristate Salt

Sitagliptin base (2 g, 0.0049 mole) was charged in isopropyl alcohol (20 ml) at 25° C. to 32° C. Myristic acid (1.12 g, 0.0049 mole) was charged at 25° C. to 32° C. to obtain a reaction mixture. The reaction mixture was heated to 50° C. and stirred for 2.5 hours at 50° C. The reaction mixture was cooled to 25° C. to 32° C. and stirred for 16 hours at 25° C. to 32° C. The reaction mixture was concentrated completely under vacuum at 50° C. Hexanes (10 ml) was charged to reaction mixture and stirred for 1 hour at 20° C. The solid was filtered and washed with isopropyl alcohol (2×10 ml). Material was dried under vacuum at 40° C. for 16 hours to obtain the titled compound.

### Yield: 2.9 g

### Example 3

### Preparation of Sitagliptin Isophthalate Salt

Sitagliptin base (2 g, 0.0049 mole) was charged in isopropyl alcohol (20 ml) at 25° C. to 32° C. Isophthalic acid (0.81 g, 0.0049 mole) was charged at 25° C. to 32° C. to obtain a reaction mixture. The reaction mixture was heated to 50° C. and stirred for 2 hours at 50° C. The reaction mixture was cooled to 25° C. to 32° C. and stirred for 16 hours at 25° C. to 32° C. to obtain a solid. The solid was filtered and washed with isopropyl alcohol (2×10 ml). The solid was dried under vacuum at 40° C. for 16 hours to obtain the titled compound.

### Yield: 2.6 g

### Example 4

### Preparation of Sitagliptin Isonicotinate Salt

Sitagliptin base (1 g, 0.00245 mole) was charged in ethanol (10 ml) at 25° C. to 32° C. Isonicotinic acid (0.3 g, 0.00245 mole) was charged at 25° C. to 32° C. The reaction mixture was heated to 60° C. and stirred for 30 minutes at 60° C. The reaction mixture was cooled to 25° C. to 32° C. and stirred for 30 minutes at 25° C. to 32° C. to obtain a solid. The solid was filtered and washed with ethanol (10 ml). The solid was dried under air oven at 40° C. for 16 hours to obtain the titled compound.

### Yield: 0.5 g

### Example 5

### Preparation of Sitagliptin Adipate

Sitagliptin base (2 g, 0.0049 moles) was charged in isopropyl alcohol (20 ml) at 25° C. to 32° C. Adipic acid (0.71 g, 0.0049 moles) was charged at 25° C. to 32° C. The reaction mixture was heated to 50° C. and stirred for 2 hours at 50° C. The reaction mixture was then cooled to 25° C. to 32° C. and distilled under vacuum completely at 50° C. to obtain a solid. The solid was dried under vacuum at 40° C. for 16 hours to obtain the titled compound.

Melting point: 99° C. to 104° C.
Yield: 2.6 g (1.3 w/w, 95.9%)

1. A salt of sitagliptin with an organic acid selected from the group consisting of 4-methylsalicylic acid, myristic acid, isophthalic acid, isonicotnic acid, and adipic acid.

2. The salt of sitagliptin according to claim 1, wherein the salt is sitagliptin 4-methyl salicylate.

3. The sitagliptin 4-methyl salicylate of claim 2, in amorphous form.

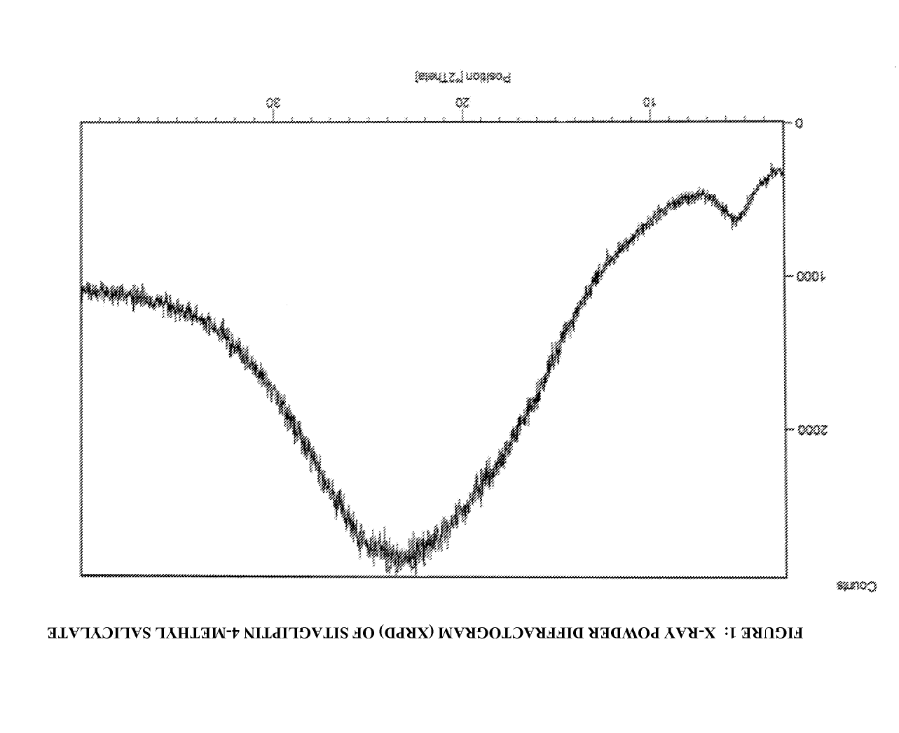

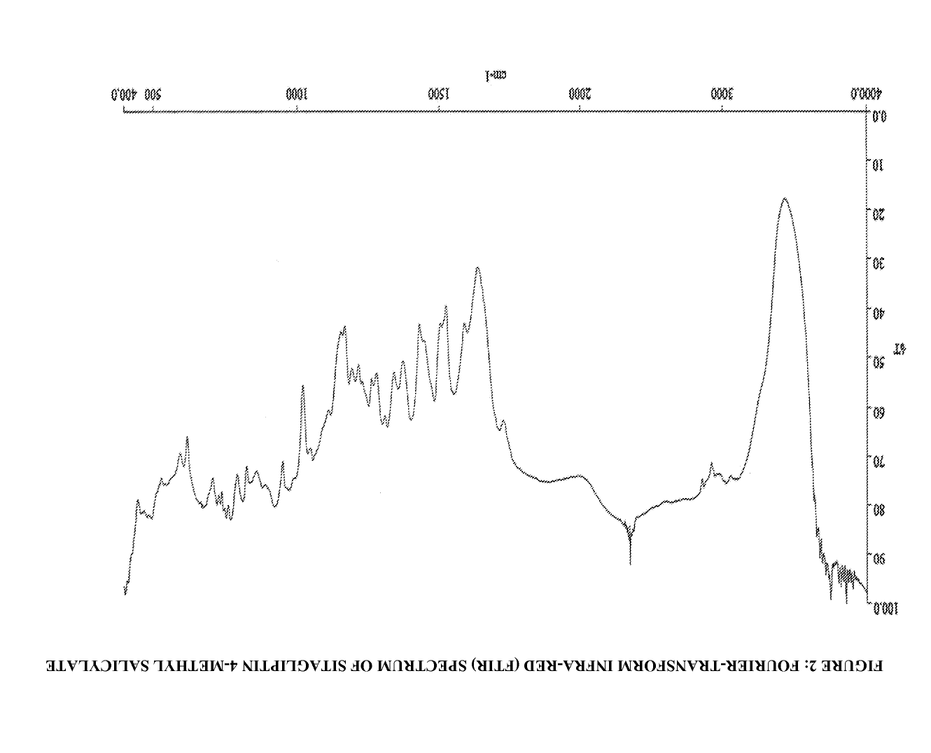

4. The sitagliptin 4-methyl salicylate of claim 3, wherein the amorphous form is characterized by an XRPD pattern substantially the same as depicted in FIG. 1.

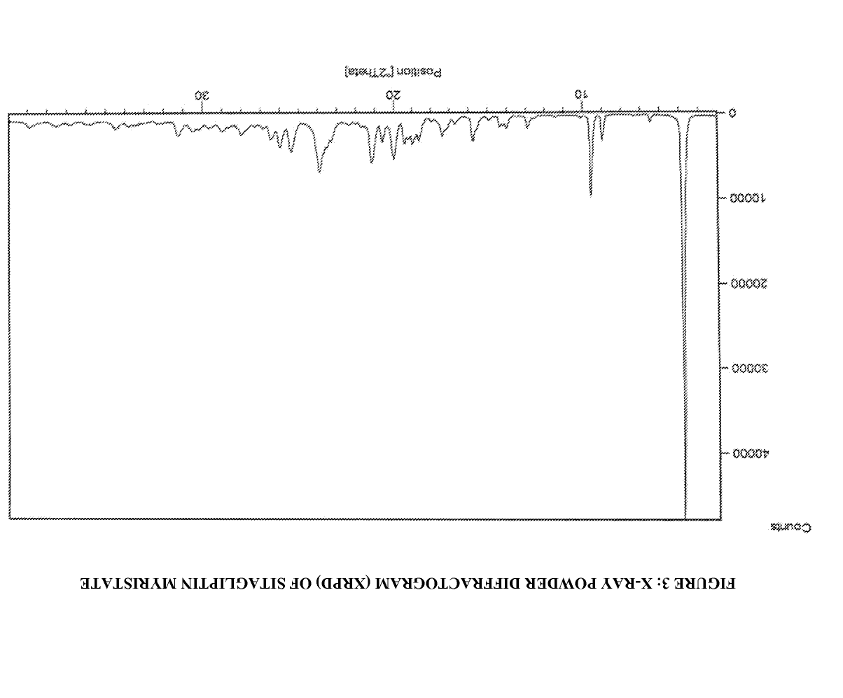

5. The sitagliptin 4-methyl salicylate of claim 3, wherein the amorphous form is characterized by FTIR as depicted in FIG. 2.

6. The salt of sitagliptin according to claim 1 wherein the salt is sitagliptin myristate.

7. The sitagliptin myristate of claim 6, in crystalline form.

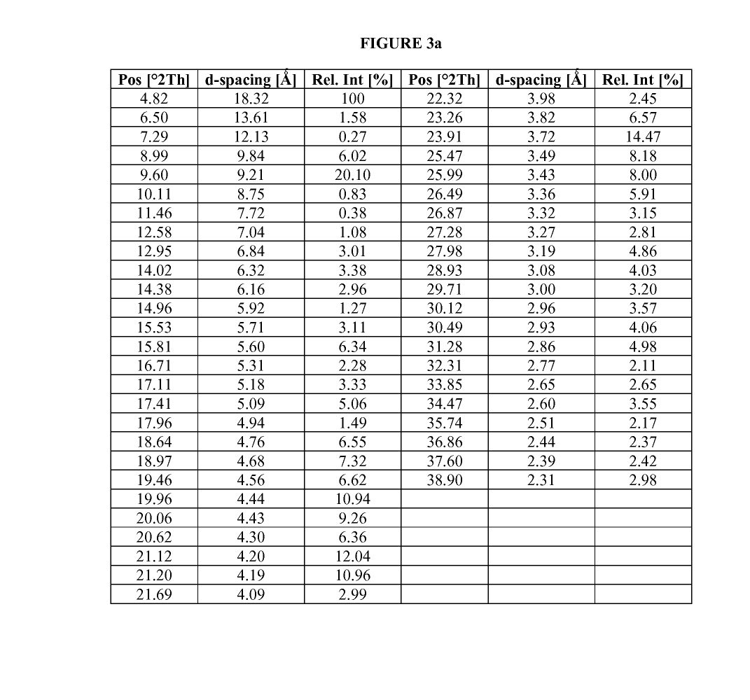

8. The sitagliptin myristate of claim 7, wherein the crystalline form is characterized by an XRPD pattern substantially the same as depicted in FIG. 3.

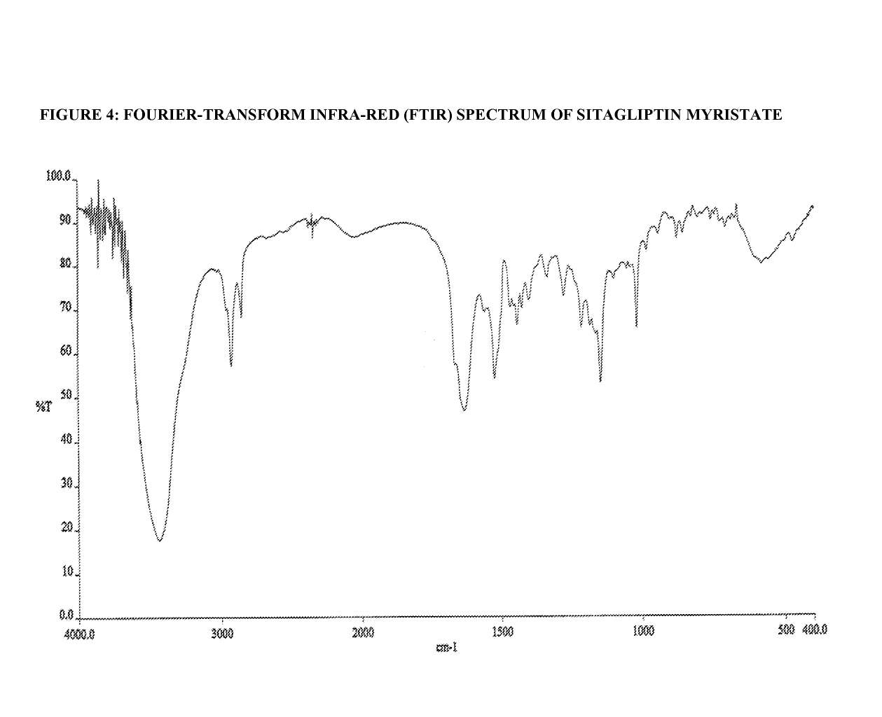

9. The sitagliptin myristate of claim 7, wherein the crystalline form is characterized by FTIR as depicted in FIG. 4.

10. The sitagliptin myristate of claim 7, wherein the crystalline form is characterized by an X-ray powder diffractogram which includes interplanar spacing (d) values substantially at about 18.31, 9.21, 4.20, 4.19, and 3.71 Å.

11. The sitagliptin myristate of claim 10, wherein the crystalline form is further characterized by an X-ray powder diffractogram which further includes additional interplanar spacing (d) values substantially at about 4.76, 4.68, 4.56, 4.43, 4.44, 4.30, 3.82, 3.49, 3.42, 3.36, and 3.18 Å.

12. The sitagliptin myristate of claim 7, wherein the crystalline form is characterized by an X-ray powder diffractogram which includes characteristic peak values (2θ) at about 4.82, 9.60, 21.11, 21.20, and 23.91±0.2°.

13. The sitagliptin myristate of claim 12, wherein the crystalline form is characterized by an X-ray powder diffractogram which further includes additional characteristic peak values (2θ) at about: 18.63, 18.97, 19.46, 19.96, 20.06, 20.62, 23.26, 25.46, 25.99, 26.48, and 27.98±0.2°.

14. The salt of sitagliptin according to claim 1, wherein the salt is sitagliptin isophthalate.

15. The sitagliptin isophthalate of claim 14, in crystalline form.

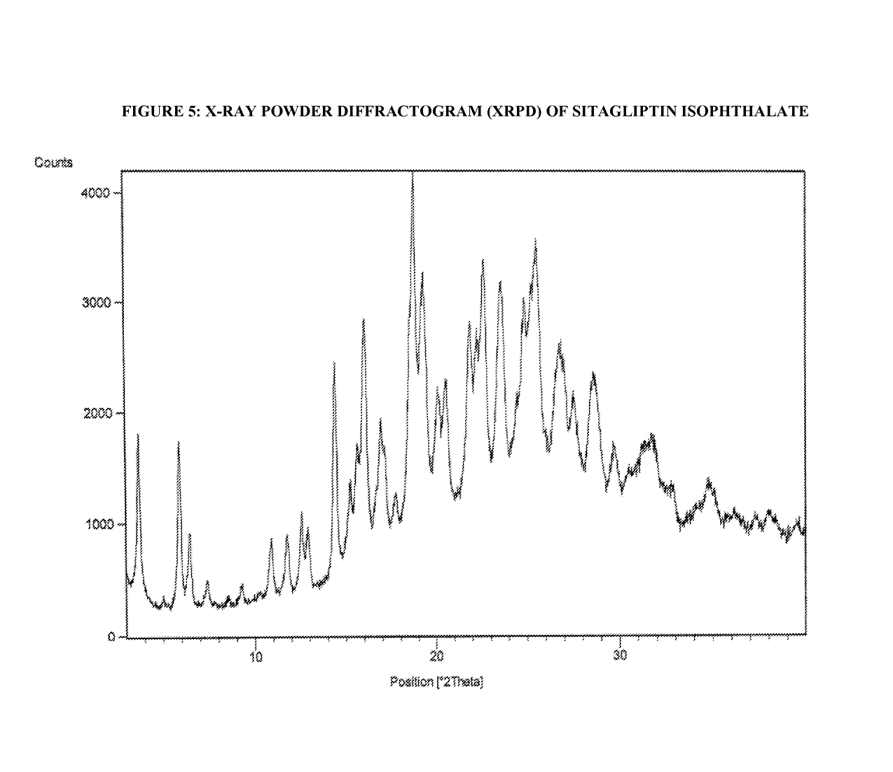

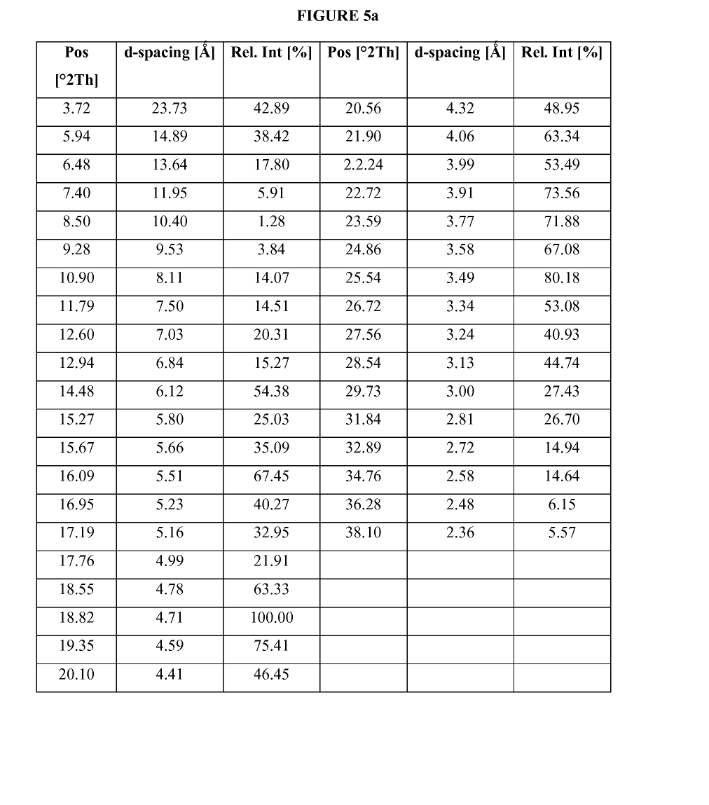

16. The sitagliptin isophthalate of claim 15, wherein the crystalline form is characterized by an XRPD pattern substantially the same as depicted in FIG. 5.

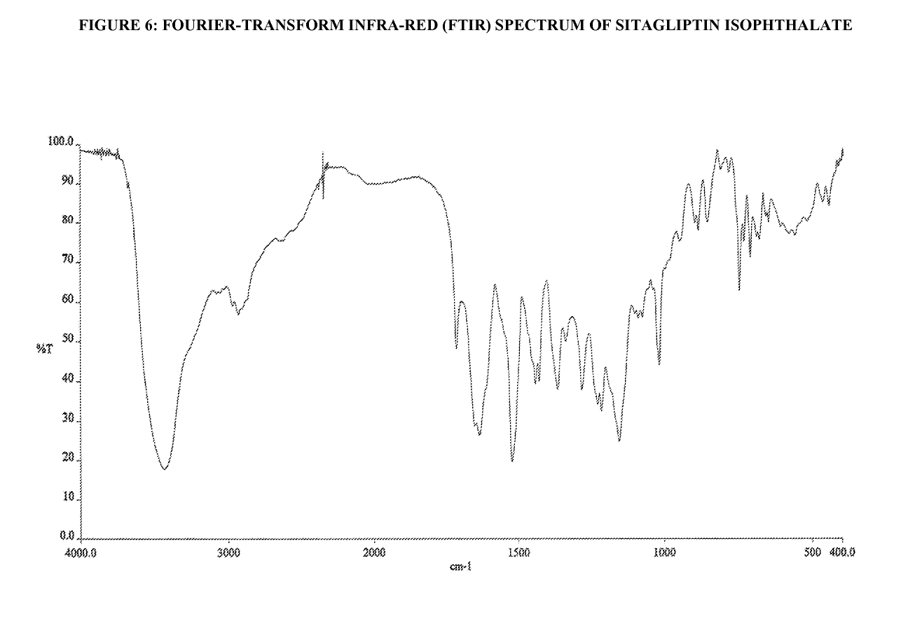

17. The sitagliptin isophthalate of claim 15, wherein the crystalline form is characterized by FTIR as depicted in FIG. 6.

18. The sitagliptin isophthalate of claim 15, wherein the crystalline form is characterized by an X-ray powder diffractogram which includes interplanar spacing (d) values substantially at about 3.49, 4.59, 3.91, 3.77, and 4.71 Å.

19. The sitagliptin isophthalate of claim 18, wherein the crystalline form is characterized by an X-ray powder diffractogram which further includes additional interplanar spacing (d) values substantially at about 23.73, 14.89, 7.03, 6.12, 5.80, 5.66, 5.51, 5.23, 5.16, 4.99, 4.78, 4.42, 4.32, 4.06, 3.99, 3.58, 3.34, 3.24, 3.13, 3.00, and 2.81 Å.

20. The sitagliptin isophthalate of claim 15, wherein the crystalline form is characterized by an X-ray powder diffractogram pattern with the following characteristic peak values (2θ) at about 18.82, 19.35, 22.72, 23.59, and 25.54±0.2°.

21. The sitagliptin isophthalate of claim 20, wherein the crystalline form is characterized by an X-ray powder diffractogram which further includes additional characteristic peak values (2θ) at about: 3.72, 5.94, 12.59, 14.48, 15.27, 15.67, 16.09, 16.95, 17.19, 17.76, 18.55, 20.10, 20.56, 21.90, 22.24, 24.86, 26.72, 27.56, 28.54, 29.73, and 31.84±0.2°.

22. The sitagliptin salt according to claim 1, wherein the salt is sitagliptin isonicotinate.

23. The sitagliptin isonicotinate of claim 22, in crystalline form.

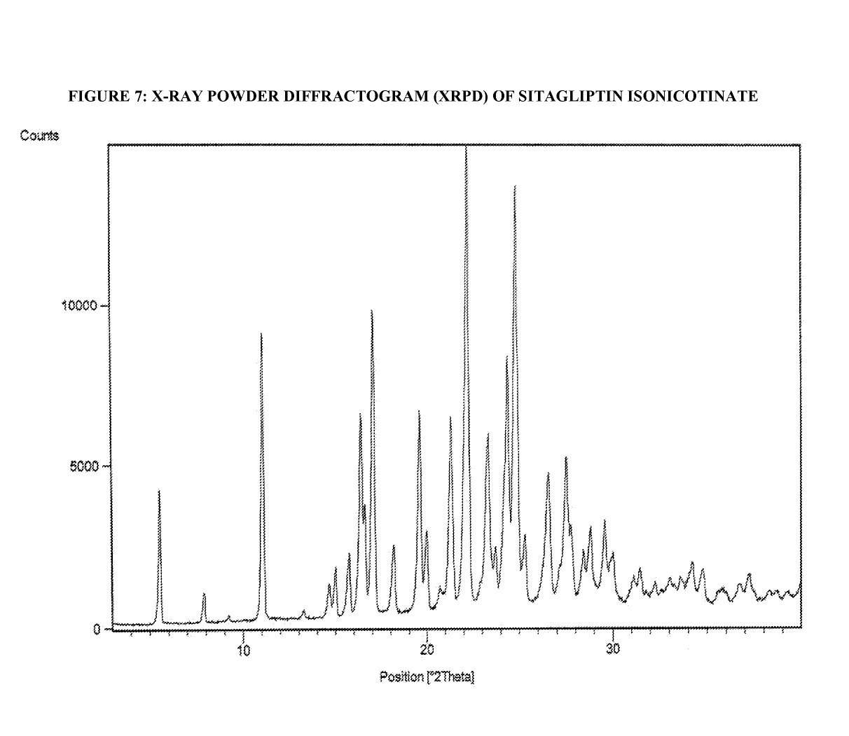

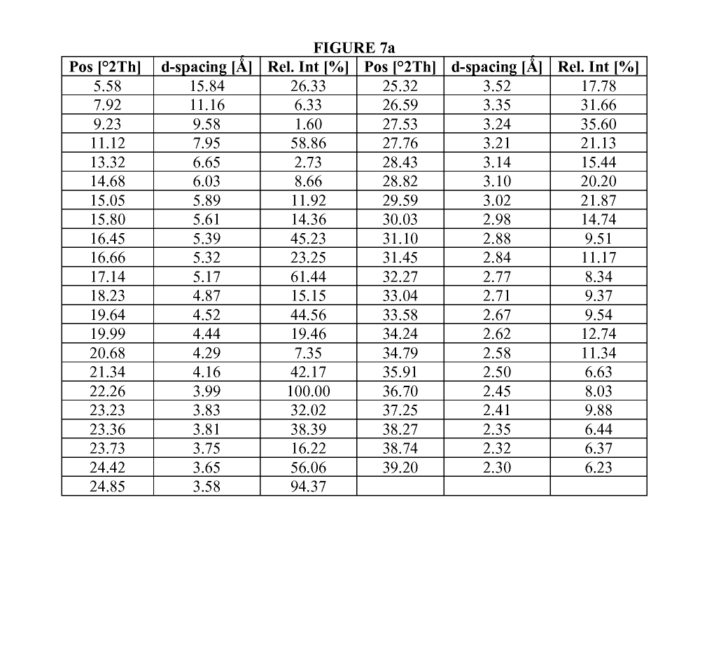

24. The sitagliptin isonicotinate of claim 23, wherein the crystalline form is characterized by an XRPD pattern substantially the same as depicted in FIG. 7.

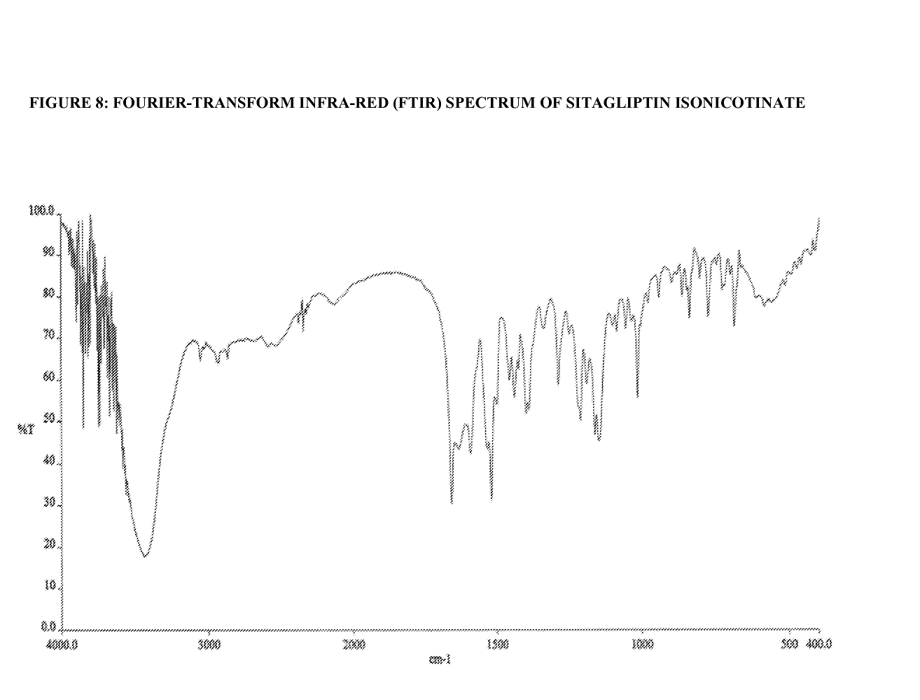

25. The sitagliptin isonicotinate of claim 23, wherein the crystalline form is characterized by FTIR as depicted in FIG. 8.

26. The sitagliptin isonicotinate of claim 23, wherein the crystalline form is characterized by an X-ray powder diffractogram having interplanar spacing (d) values substantially at about 7.95, 5.17, 3.99, 3.65, and 3.58 Å.

27. The sitagliptin isonicotinate of claim 26, wherein the crystalline form is characterized by an X-ray powder diffractogram which further includes additional interplanar spacing (d) values substantially at about 15.84, 5.39, 5.32, 4.52, 4.16, 3.83, 3.81, 3.35, and 3.24 Å.

28. The sitagliptin isonicotinate of claim 23, wherein the crystalline form is characterized by an X-ray powder diffractogram pattern with the following characteristic peak values (2θ) at about: 11.12, 17.14, 22.26, 24.41, and 24.85±0.2°.

29. The sitagliptin isonicotinate of claim 23, wherein the crystalline form is characterized by an X-ray powder diffractogram further including additional characteristic peak values (2θ) at about: 5.58, 16.45, 16.66, 19.64, 21.34, 23.23, 23.36, 26.59 and 27.53±0.2°.

30. The sitagliptin salt according to claim 1, wherein the salt is sitagliptin adipate.

31. The sitagliptin adipate of claim 30, in crystalline form.

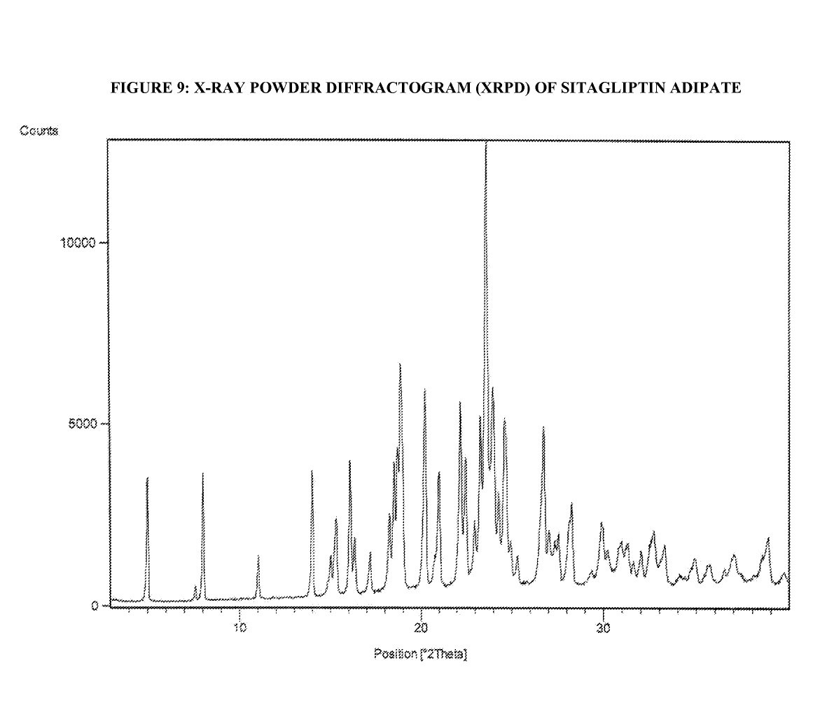

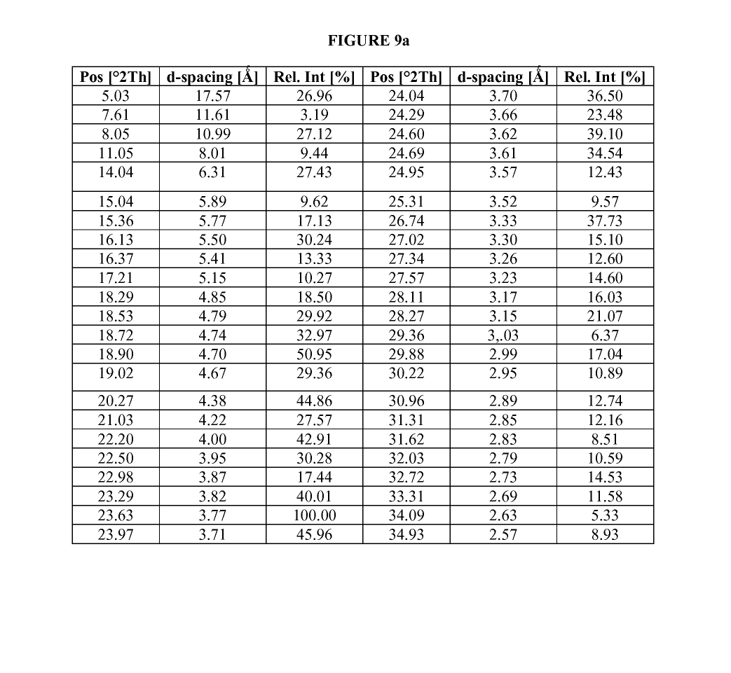

32. The sitagliptin adipate of claim 31, wherein the crystalline form is characterized by an XRPD pattern substantially the same as depicted in FIG. 9.

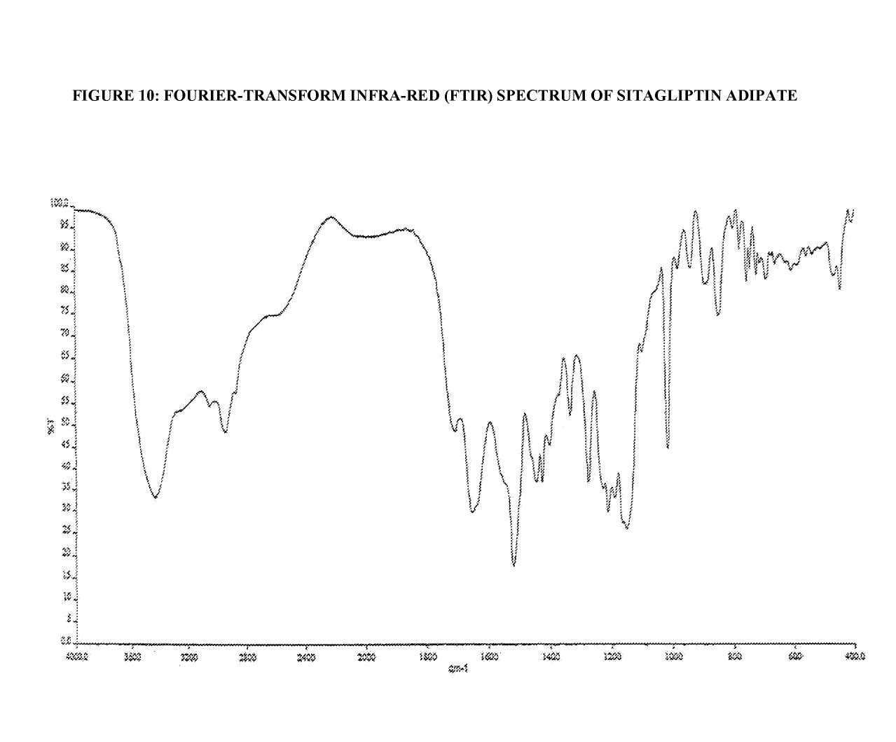

33. The sitagliptin adipate of claim 31, wherein the crystalline form is characterized by FTIR as depicted in FIG. 10.

34. The sitagliptin adipate of claim 31, wherein the crystalline form is characterized by an X-ray powder diffractogram having interplanar spacing (d) values substantially at about t 4.70, 4.38, 4.00, 3.82, 3.77 and 3.71 Å.

35. The sitagliptin adipate of claim 31, wherein the crystalline form is characterized by an X-ray powder diffractogram further including additional interplanar spacing (d) values substantially at about 17.57, 10.99, 6.31, 5.50, 4.79, 4.74, 4.22, 3.95, 3.70, 3.62 and 3.33 Å.

36. The sitagliptin adipate of claim 31, wherein the crystalline form is characterized by an X-ray powder diffractogram pattern with the following characteristic peak values (2θ) at about: 18.90, 20.27, 22.20, 23.29, 23.63 and 23.97±0.2°.

37. The sitagliptin adipate of claim 31, wherein the crystalline form is characterized by an X-ray powder diffractogram further including additional characteristic peak values (2θ) at about: 5.03, 8.05, 14.04, 16.13, 18.53, 18.72, 21.03, 22.50, 24.04, 24.60 and 26.74±0.2°.

38. A process for the preparation of a compound of Formula 1 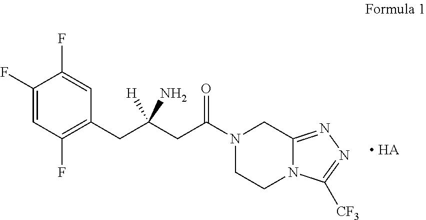
the process comprising: treating sitagliptin or its salt and HA, wherein HA is selected from the group consisting of 4-methylsalicylic acid, myristic acid, isophthalic acid, isonicotinic acid or adipic acid.

39. A process according to claim 38, wherein sitagliptin or its salt is treated with HA directly or in the presence of a suitable solvent at a suitable temperature.

40. A process according to claim 39, wherein the solvent includes water, esters, alkanols, halogenated hydrocarbons, ketones, ethers, polar aprotic solvents, or mixtures thereof.

41. (canceled)

42. (canceled)

43. (canceled)

44. (canceled)

45. (canceled)

46. (canceled)

47. A process according to claim 39, wherein the sitagliptin or its salt is treated with HA at a temperature of about 30° C. to reflux.

48. A process for the preparation of sitagliptin, salts, solvates or polymorphs thereof, which includes the use of a compound of Formula 1 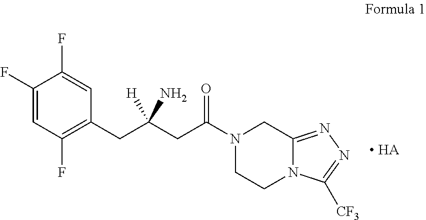
wherein HA is selected from the group consisting of 4-methylsalicylic acid, myristic acid, isophthalic acid, isonicotinic acid or adipic acid.

49. (canceled)

50. A process according to claim 42, wherein the process includes contacting the compound of Formula 1 with a base wherein the base is selected from a group comprising hydroxides, carbonates and bicarbonates of alkali and alkaline earth metals, ammonia, alkyl amines and hydrazine.

51. (canceled)

52. A pharmaceutical composition comprising at least one salt selected from the group consisting of sitagliptin 4-methyl salicylate, sitagliptin myristate, sitagliptin isophthalate, sitagliptin isonicotinate and sitagliptin adipate, and a pharmaceutically acceptable carrier.

53. A method of treating or preventing type 2 diabetes mellitus which comprises administering to a patient in need thereof a therapeutically effective amount of the pharmaceutical composition according to claim 44.
